SEQUENCE LISTING (4 sequences)

SEQ ID NO: 1 - protein, 585 aa, Homo sapiens [sequence omitted]

SEQ ID NO: 2 - protein, 41 aa, Homo sapiens
LVRPEVDVMCTAFHDNEETFLKAAFTECCQAADKAACLLPK

SEQ ID NO: 3 - protein, 53 aa, Homo sapiens
KGEEAFCTEKLTAVTCLKDGFLTHLSKDCNEASEDAVCTKAYCEHPDAAACCK

SEQ ID NO: 4 - protein, 65 aa, Homo sapiens
VTKCCTESLVNRRPCFSALEVDETYVPKLAKTYETTLEKCCAAADPHECYAKTCVADESAENCDK

(19) United States
(12) Patent Application Publication
(10) Pub. No.: US 2023/0348566 A1
(43) Pub. Date: Nov. 2, 2023

(54) RE-FOLDED HUMAN SERUM ALBUMIN AND USE THEREOF FOR ANTI-TUMOR

(71) Applicant: Academia Sinica, Taipei (TW)

(72) Inventors: Chi-Ming LIANG, Taipei (TW); Jeng-Jer SHIEH, Taichung (TW); Chung-Hsuan CHEN, Taipei (TW); Shu-Mei LIANG, Taipei (TW)

(21) Appl. No.: 18/025,637

(22) PCT Filed: Sep. 10, 2021
(86) PCT No.: PCT/US2021/049816

§ 371 Date: Mar. 9, 2023

Related U.S. Application Data
(60) Provisional application No. 63/077,585, filed on Sep. 12, 2020.

(51) Int. Cl. C07K 14/765; A61P 35/00

(52) U.S. Cl. CPC C07K 14/765; A61P 35/00

(57) ABSTRACT

Re-folded human serum albumin (rfHSA) and use thereof for anti-tumor are disclosed. The rfHSA comprises the primary amino acid sequence of naive human serum albumin, in which the rfHSA in a solution is oval shape, not fibrillar, and the naive HSA is globular. The rfHSA is used for treating cancer or a tumor in a subject in need thereof The rfHSA may also be used as a reagent for detecting the presence of a cancer cell associated with integrin β1 or serine/threonine protein kinase Akt and extracellular signal-regulated kinase 1/2 (ERK1/2) in a tumor sample or as a reagent for inhibiting phosphorylation of Akt and ERK 1/2 in a cancer cell sample. A cell lysate of a cancer cell treated with rfHSA, a vaccine composition comprising the cancer cell lysate, and use thereof are also disclosed. Also disclosed is a method tor preparing rfHSA.

### FIELD OF THE INVENTION

The present invention relates generally to re-folded human serum albumin with anti-tumor activities.

### BACKGROUND OF THE INVENTION

U.S. Pat. Nos. 8,357,652 and 9,226,951 disclose fibrillar human serum albumin that can cause apoptosis in many types of cancer cells by modulating the Akt signaling pathway, the contents of which are herein incorporated by reference in their entireties. Although the formation of fibrillar human serum albumin from naïve human serum albumin could be demonstrated, separating these two albumins apart to verify the purity and consistency of fibrillar human serum albumin in each production batch was not feasible by using the methods disclosed therein. Fibrillar proteins have been known to be more antigenic, therefore, fibrillar human serum albumin might be more antigenic to some subjects and cause undesirable side effects during clinical use.

### SUMMARY OF THE INVENTION

In one aspect, the invention relates to a re-folded human serum albumin (rfHSA) molecule, which comprises the primary amino acid sequence of naïve human serum albumin (naïve HSA), wherein the rfHSA molecule in a solution is oval shape, not fibrillar, and the naïve ISA is globular.

In another aspect, the invention relates to use of a rfHSA molecule, a pharmaceutical composition, or a vaccine composition of the invention in the manufacture of a medicament for treating cancer or for treating a tumor in a subject in need thereof.

In another aspect, the invention relates to use of a rfHSA molecule of the invention in the manufacture of a reagent for detecting the presence of a cancer cell that is associated with integrin β1 or serine/threonine protein kinase Akt and extracellular signal-regulated kinase 1/2 (ERK 1/2) in tumor cells or in a tumor sample.

In another aspect, the invention relates to use of a rfHSA molecule of the invention in the manufacture of a reagent for inhibiting phosphorylation of Akt and ERK1/2 in a sample comprising a cancer cell.

In another aspect, the invention relates to a kit comprising a rfFHSA molecule of the invention for detecting the presence of a cancer cell that is associated with integrin β1 or Akt and ERK1/2 in a tumor sample.

Further in another aspect, the invention relates to a cell lysate of a cancer cell treated with a rfHSA molecule of the invention.

Further in another aspect, the invention relates to a vaccine composition comprising the cell lysate of the invention.

Yet in another aspect, the invention relates to a method for preparation of a rfHSA molecule of the invention, the method comprising:
(a) dissolving human serum albumin (HSA) in a buffer solution comprising a detergent to obtain a detergent-treated HSA solution;
(b) sonicating the detergent-treated HSA solution to obtain a sonicated, detergent-treated HSA solution;
(c) subjecting the sonicated, detergent-treated HSA solution to a size exclusion chromatography column with a molecular weight range between 10,000 and 600,000 Daltons (Da);
(d) eluting the column with an eluent comprising the detergent;
(e) collecting column eluate fractions comprising the detergent-treated HSA;
(f) pooling the column eluate fractions to obtain a pooled column eluate;
(g) performing dialysis by subjecting the pooled column eluate to a dialysis membrane with molecular weight-cutoff (MWCO) of 12,000-14,000 Da against a dialysate comprising no detergent;
(h) collecting a dialysis membrane eluate;
(i) concentrating and dialyzing the dialysis membrane eluate against the dialysate comprising not detergent to obtain a concentrated, dialysis membrane eluate; and
(j) repeating the concentrating and dialyzing step (i) to obtain a final concentrated, dialysis membrane eluate comprising the rfHSA of the invention, wherein the final concentrated, dialysis membrane eluate in step (j) comprises no or little detergent.

### BRIEF DESCRIPTION OF THE DRAWINGS

FIGS. 1A-B are docking models showing the structures of (A) rfHSA and (B) naïve HSA, respectively, in a solution analyzed by biological small angel x-ray scattering. (A) Model docking of 1e78.pdb crystal structure and rfHSA envelope by SUPCOMB, showing the shape of rfHSA is oval; (B) Model docking of 1e78.pdb crystal structure and naïve HSA envelope by SUPCOMB, showing the shape of naive HSA is a globular shape.

FIGS. 2A-B are mass spectra of (A) naïve HSA (globular HSA, or gHSA) and (B) rfHSA, respectively, analyzed using liquid chromatography-tandem mass spectrometry (LC-MS-MS) after limited proteolysis under reducing condition.

FIGS. 3A-B are mass spectra of (A) naïve HSA and (B) rfHSA, respectively, analyzed using LC-MS-MS after limited proteolysis under non-reducing condition.

FIG. 4A shows the sequence of a 41 amino acid peptide (SEQ ID NO: 2) corresponding to a mass spectrum fragment ion at mass-to-charge ratio (m/z) 4559, which is present in naïve HSA but absent in rfHSA.

FIG. 4B shows the sequence of naïve HSA (SEQ ID NO: 1) and intramolecular disulfide bridges Cys124-Cys168 and Cys169-Cys177.

FIG. 5A shows the sequence of a 53 amino acid polypeptide (SEQ ID NO: 3) corresponding to a mass spectrum fragment ion at m/z 5729, which is present in naïve HSA but absent in rfHSA.

FIG. 5B shows the sequence of naïve HSA (SEQ ID NO: 1) and intramolecular disulfide bridges Cys62-Cys361 and Cys75-Cys567.

FIG. 6A shows the sequence of a 65 amino acid polypeptide (SEQ ID NO: 4) corresponding to a mass spectrum fragment ion at m/z 7223, which is present in naïve HSA but absent in rfHSA.

FIG. 6B shows the sequence of naïve HSA (SEQ ID NO: 1) and intramolecular disulfide bridges Cys62-Cys361 and Cys360-Cys487.

FIG. 7 shows the cytotoxic effects of rfHSA on a variety of cancer cells;

FIG. 8 shows the cytotoxic effects of rfHSA on clinically relevant ovarian cancer cell types.

FIG. 9A is a schematic drawing showing a treatment regimen in ovarian cancer cell bearing mice.

FIG. 9B is a set of fluorescent images showing tumor size in control (left panel) and rfHSA treatment (right panel) groups. Ms1, Ms2, Ms3 refer to mice 1, 2, and 3, respectively

FIG. 9C is a pair of graphs showing body weights (left panel) and bioluminescence imaging (BLI) levels (right panel) of ovarian cancer cell bearing mice.

FIG. 10 is a graph showing the cytotoxic effects of rfHSA on human pancreatic cancer cell lines BxPC-3, MIA PaCa-2, and PANC-1.

FIG. 11 is a western blot image showing that rfHSA suppressed the phosphorylation of Akt and ERK and the inhibitory effects of rfHSA was reversed by anti-integrin antibodies in BxPC-3.

FIG. 12 is a graph showing that rfHSA does not affect the viability of normal cells human primary peripheral blood mononuclear cells.

FIG. 13 is a bar graph showing the cytotoxic effect of rfHSA on B16F10 melanoma cells.

FIG. 14A is a schematic drawing showing a vaccination regimen and melanoma cancer cell challenge in mice. The mice were vaccinated with lysate of rfHSA-treated B16F10 melanoma cells.

FIG. 14B is a graph showing the tumor size of the mice in FIG. 14A.

FIG. 14C is a graph showing the survival rate of the mice in FIG. 14A.

FIG. 14D is a graph showing tumor free mice percentage in FIG. 14A.

### DETAILED DESCRIPTION OF THE INVENTION

### Definitions

Unless otherwise defined, all technical and scientific terms used herein have the same meaning as commonly understood by one of ordinary skill in the art to which this invention pertains. In the case of conflict, the present document, including definitions will control.

The term “globular” means spherical; having the shape of a sphere or ball.

The term “oval” means having the general form, shape, or outline of an egg; egg-shaped.

The term “fibrillar” means relating to a fibril. A fibril is a small or fine fiber or filament; or a threadlike structure or filament. A filament is a very fine thread or threadlike structure.

The term “polypeptide or peptide fragment” refers to a polypeptide or peptide that has an amino-terminal or carboxy-terminal deletion, but where the remaining amino acid sequence is identical to the corresponding positions in the naturally-occurring sequence deduced, for example, from a full-length cDNA sequence. Fragments typically are at least 5, 6, 8 or 10 amino acids long, preferably at least 14 amino acids long, more preferably at least 20 amino acids long, usually at least 50 amino acids long, and even more preferably at least 70 amino acids long.

rfHSA can be included in a pharmaceutical composition together with additional active agents and pharmaceutically acceptable carriers, vehicles, excipients, or auxiliary agents.

The term “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, intraperitoneal, intraarterial, intramuscular, intralesional, and rectal administration.

“Subject” as used herein refers to humans and non-human animals.

As used herein, the terms “naïve human serum albumin”, “globular human serum albumin” are interchangeable.

An arginine-glycine-aspartic acid (RGD) motif is a cell adhesion motif. It was originally identified as the sequence within fibronectin that mediates cell attachment. The family of membrane proteins known as integrins act as receptors for these cell adhesion molecules via the RGD motif.

The term “Akt” refers to “serine/threonine protein kinase Akt (protein kinase B)”. The Akt signaling pathway or PI3K-Akt signaling pathway is a signal transduction pathway that promotes survival and growth in response to extracellular signals. Key proteins involved are PI3K (phosphatidylinositol 3-kinase) and Akt (protein kinase B).

A composition comprising a rfHSA molecule of the invention may be prepared with carriers that will protect the active ingredient against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to cell-specific antigens) can also be used as pharmaceutically acceptable carriers.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED.sub.50 (the dose therapeutically effective in 50% of the population). The dose ratio between toxic and therapeutic effects is the therapeutic index and it can be expressed as the ratio LD₅₀/ED₅₀ Compounds which exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage can vary within this range depending upon the dosage form employed and the route of administration utilized. For any compound used in the method of the disclosure, the therapeutically effective dose can be estimated initially from cell culture assays. A dose can be formulated in animal models to achieve a circulating plasma concentration range that includes the IC.sub.50 (i.e., the concentration of the test compound which achieves a half-maximal inhibition of symptoms) as determined in cell culture. Such information can be used to determine useful doses more accurately in humans. Levels in plasma can be measured, for example, by high performance liquid chromatography.

The term “treating”, or “treatment” refers to administration of an effective amount of a therapeutic agent to a subject in need thereof with the purpose of cure, alleviate, relieve, remedy, or ameliorate the disease. Such a subject can be identified by a health care professional based on results from any suitable diagnostic method.

“An effective amount” refers to the amount of an active agent that is required to confer a therapeutic effect on the treated subject. Effective doses will vary, as recognized by those skilled in the an, depending on routes of administration, excipient usage, and the possibility of co-usage with other therapeutic treatment.

The term “chemotherapeutic agent” refers to a pharmacological agent that is known to be of use in the treatment of cancer.

The “Guidance for Industry and Reviewers Estimating the Safe Starting Dose in Clinical Trials for Therapeutics in Adult Healthy Volunteers” published by the U.S. Department of Health and Human Services Food and Drug Administration discloses a “therapeutically effective amount” may be obtained by calculations from the following formula:

HED=animal dose in mg/kg×(animal weight in kg/human weight in kg)^{0.33}.

The body weight of mice used in the illustrated study below ranges from 16-22 grams.

Abbreviations: naïve human serum albumin, naïve HSA; globular human serum albumin, gHSA; re-folded human serum albumin, rfHSA; an arginine-glycine-aspartic acid, RGD; liquid chromatography-tandem mass spectrometry, LC-MS-MS; mass-to-charge ratio, m/z; Bioluminescence Imaging, BLI: room temperature, RT.

Sequence listing: naïve human serum albumin (SEQ
ID NO: 1);
(SEQ ID NO: 2; 41 aa)
LVRPEVDVMCTAFHDNEETFLKAAFT ECCQAADKAA CLLPK;
(SEQ ID NO: 3: 53 aa)
KGEEAFCTEKLTAVTCLKDGFLTHLSKDCNEASEDAVCTKAYCEHPDAAA
CCK;
(SEQ ID NO: 4; 65 aa)
VTKCCTESLVNRRPCFSALEVDETYVPKLAKTYETTLEKCCAAADPHECY
AKTCVADESAENCDK.

The invention provides an anti-cancer re-folded human serum albumin (rfHSA) and methods of making and using the same. The rfHSA is a monomer, comprising the same primary sequence as naïve HSA. The method used for preparing the rfHSA has advantages including ease of purity verification, consistency of production, and feasibility of scaling up.

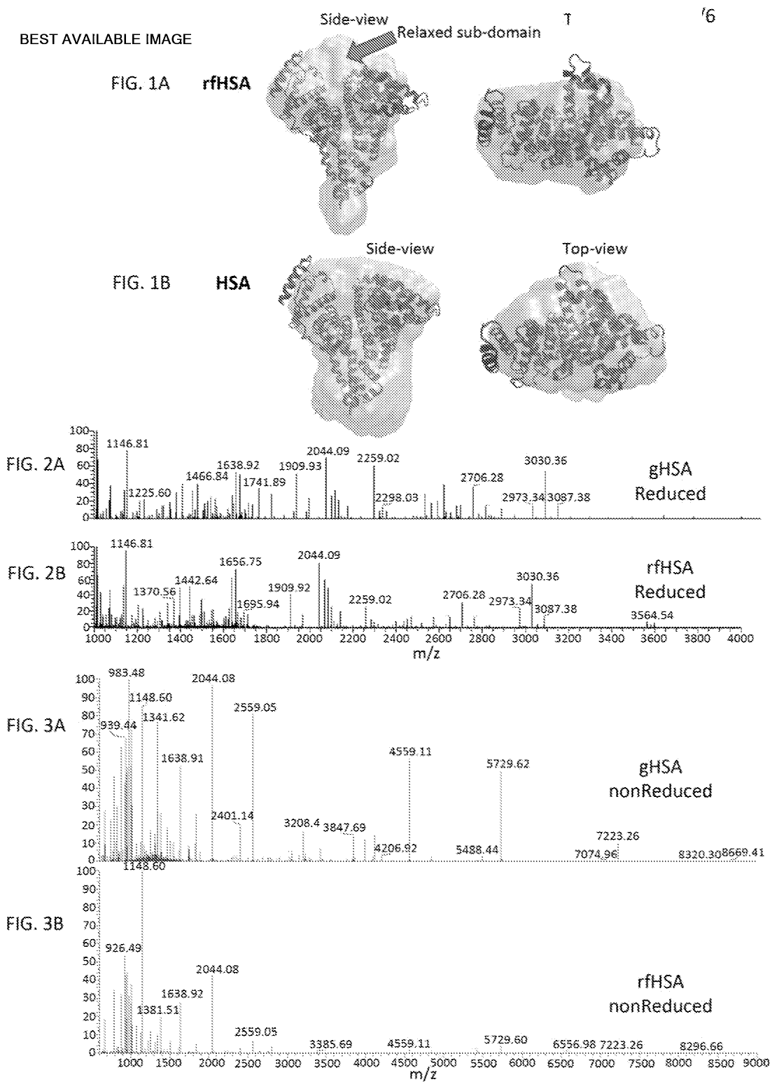

Biological small angel x-ray scattering indicates that rfHSA is oval shape rather than globular shape of naïve HSA (FIG. 1). The rfHSA is distinguishable from naïve HSA in that at least 3 of the 17 intramolecular disulfide bonds of naïve HSA are disrupted in rfHSA, as evidenced by limited proteolysis followed by liquid chromatography-tandem mass spectrometry (LC-MS-MS) analysis. LC-MS-MS analysis of rfHSA after trypsin limited proteolysis under non-reducing condition shows the absence of several major fragment ions, notably, the fragment ions at m/z 4559, 5729 and 7223 (FIGS. 2-3).

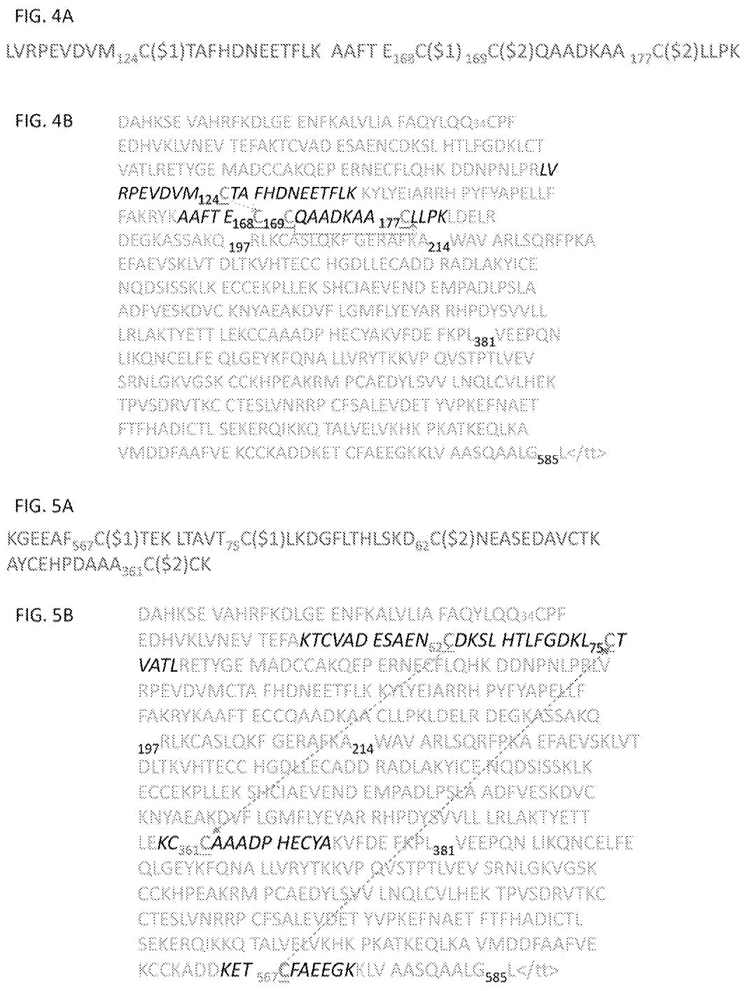

A 41 amino acid (aa) peptide with the sequence of LVRPEVDVMC($1)TAFHDNEETFLKAAFTEC($1)C($2)QAADKAA C($2)LLPK (SEQ ID NO: 2), corresponding to the fragment ion at m/z 4559, is present in naïve HSA but absent in rfHSA. The 41 aa peptide is formed by linking cysteine 124 to cysteine 168 and cysteine 169 to cysteine 177 of HSA (FIG. 4).

A 53 amino acid polypeptide with the sequence of KGEEAFC($1)TEKLTAVTC($1)LKDGFLTHLSKDC($2)NEASEDAVCTKAYCEHPDAAAC($2)CK (SEQ ID NO: 3), corresponding to the fragment ion at m/z 5729, is present in naïve HSA but absent in rfHSA. The 53 aa peptide is formed by linking cysteine 567 to cysteine 75 and cysteine 62 to cysteine 361 (FIG. 5).

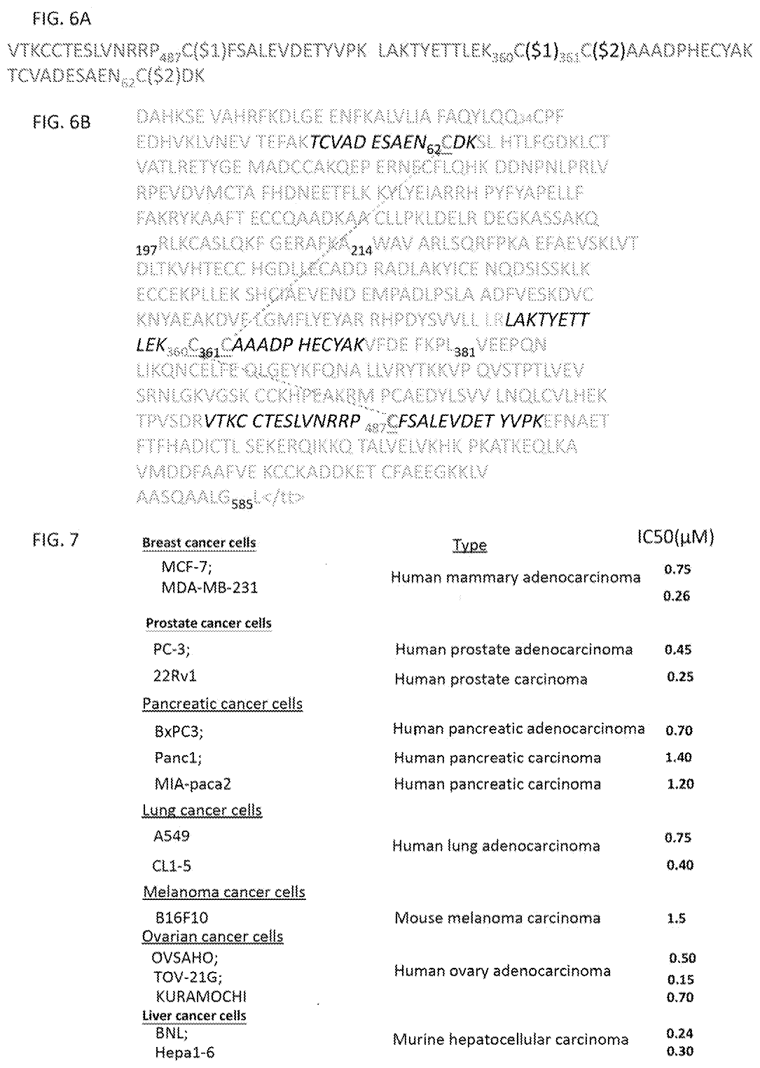

A 65 amino acid polypeptide with the sequence of VTKCCTESLVNRRPC($1)FSALEVDETYVPKLAKTYETTLEKC ($1)C($2)AAADPHECYAKTCVADESAENC($2)DK (SEQ ID NO: 4), corresponding to fragment ion at m/z 7223, is present in naïve HSA but absent in rfHSA. The 65 aa is formed by linking cysteine 487 to cysteine360 and cysteine 361 to cysteine 62 (FIG. 6).

Naïve HSA has been unexpectedly converted into rfHSA after SDS being exhaustively removed (preferably to a level of ≤0.18 mg SDS/mg rfHSA) during the processes for creating fibrillar human serum albumin (HSA). Methods for creating a fibrillar HSA is disclosed in U.S. Pat. No. 9,226,951, which is herein incorporated by reference in its entirety.

In one embodiment of the invention, a rfHSA was generated by dissolving naïve HSA in a 1% SDS solution, passing through a SUPERDEX®-200 gel filtration column and eluting with a dialysate solution containing 25 mM Tris-HCl (pH 8.0), 1 mM EDTA, 0.1 M NaCl, and 0.05% SDS. After dialysis against dialysate, the eluate inside dialysis tubing was collected, concentrated and dialyzed against dialysate again. The same procedure was repeated several times to remove the SDS as exhaustively as possible. It was found that unlike fibrillar HSA, the rfHSA did not form fibrillar form.

The rfHSA has cytotoxic effect on a variety of cancer cells, with potency about the same magnitude as fibrillar HSA. The advantage of using rfHSA instead of fibrillar HSA as an anti-cancer agent is that rfHSA is not a fibrillar protein. Fibrillar ISA might be more antigenic to some subjects and can cause undesirable side effects during clinical use. In addition, the purity and consistency of rfHSA is more verifiable than fibrillar HSA.

In one embodiment, rfHSA is used for treating kidney, breast, lung, prostate, liver, melanoma, or ovarian cancer (FIG. 7).

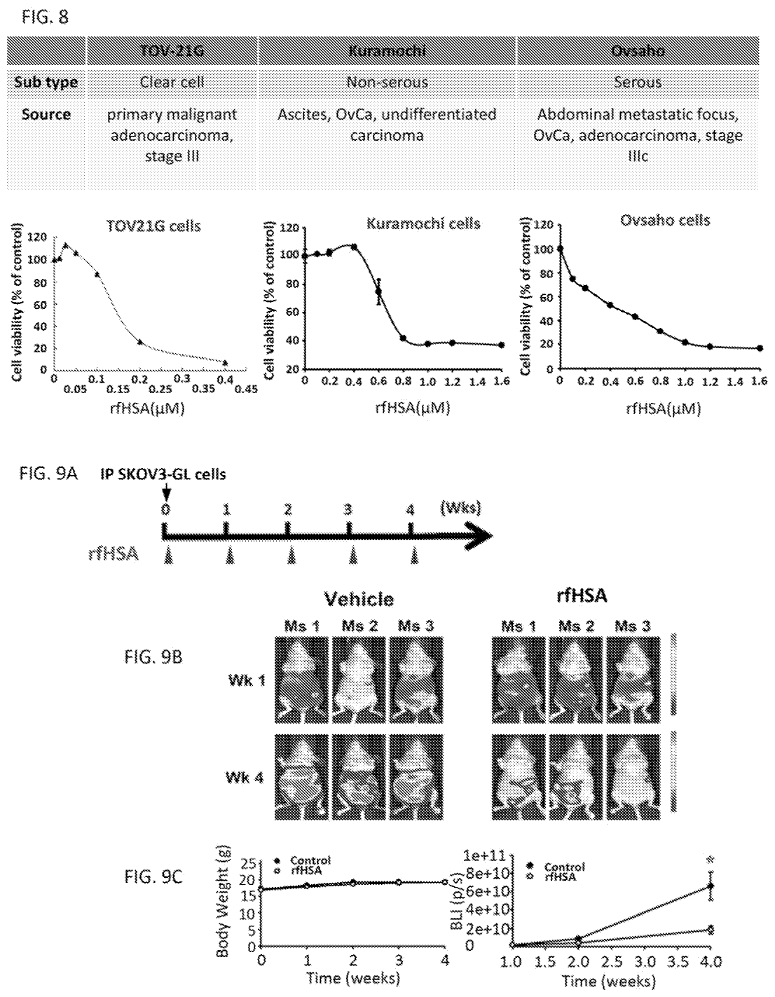

The cytotoxic effect and IC₅₀ of rfHSA on clinically relevant ovarian (TOV21G, KURAMOCHI, OVSAHO), pancreatic (BxPC3, MIA-paca2 and Panc1), and melanoma (B16F10) cancer cells are shown in FIGS. 8, 10 and 13, respectively. FIG. 9 shows in vivo anti-cancer effect of rfHSA on ovarian cancer bearing mice.

rfHSA inhibits phosphorylation of Akt and ERK in an integrin dependent manner in pancreatic cancer cells (BxPC3) (FIG. 11). As rfHSA bound to the receptors such as integrins on the cell surface while globular serum albumin could not, it is believed that the change in the structure of serum albumin from globular to re-folded form has enabled the proteins to selectively target cancer cells that expressed more integrin.alpha.5.beta.1 than normal cells.

Normal cells (human peripheral blood mononuclear cells) were treated with rfHSA (0-1.6 μM) for 24-72 hrs. Little effects on the viability of the normal cells were detected (FIG. 12).

The lysate of rfHSA-treated cancer cells may be used as a vaccine for cancer bearing subjects. The cancer cells may be B16F10 melanoma.

The rfHSA protein, variant, derivate, ortholog, or other protein having substantial identity to human serum albumin for treating the cancer may be selected based on the severity of the disease and the desired cytotoxicity to the cancer cells.

For greater cytotoxicity to the cancer cells, a protein with an RGD motif or greater molecular weight is selected. RGD motif is a ligand for integrins. It has been shown that re-folded proteins induced cell death via modulating integrin/Akt signaling pathway. It has been found that re-folded proteins with RGD motifs, like rVP1-S200 and FN-S200, were more cytotoxic than those without RGD motifs such as bovine serum albumin.

In one aspect, the invention provides a re-folded human serum albumin (rfHSA), which comprises the primary amino acid sequence of naïve human serum albumin (naïve HSA), wherein the rfHSA in a solution is oval shape, not fibrillar, and the naïve HSA is globular.

In one embodiment, the rfHSA of the invention lacks two or more intramolecular disulfide bridges selected from the group consisting of: (i) C124-C168 and C169-C177; (ii) C567-C75 and C62-C361; (iii) C487-C360 and C361-C62; and (iv) any combination thereof.

In another embodiment, the rfHSA of the invention lacks the intramolecular disulfide bridges C124-C168, C169-C177, C567-C75, C62-C361, and C487-C360.

In another embodiment, the rfHSA of the invention after limited trypsin proteolysis under nonreduced conditions lacks mass spectrum fragment ions at mass to charge ratios (m/z) of 4559, 5729 and 7223 that are present in the naïve HSA.

In another embodiment, the rfHSA of the invention after limited trypsin proteolysis under nonreduced conditions lacks mass spectrum fragment ions at mass to charge ratios (m/z) of 4559, 5729 and 7223, wherein the fragment ions at the m/z of 4559, 5729 and 7223t are present after the limited trypsin proteolysis of the naïve HSA.

In another embodiment, the rfHSA of the invention after limited trypsin proteolysis under nonreduced conditions generates peptide fragments, the generated fragments lacking one or more peptide fragments that are selected from the group consisting of SEQ ID NOs: 2, 3, 4, and any combination thereof, wherein the lacked one or more peptide fragments are present after the limited trypsin proteolysis of the naïve HAS.

In another embodiment, the rfHSA of the invention after limited trypsin proteolysis under nonreduced conditions generates peptide fragments, the generated fragments lacking peptide fragments comprising the amino acid sequence of SEQ ID NOs: 2, 3, and 4, wherein the lacked peptide fragments comprising the SEQ ID NOs: 2, 3, and 4 are present after the limited trypsin proteolysis of the naïve HAS.

The invention also provides a pharmacological composition comprising a rfHSA of the invention and a pharmaceutically acceptable carrier, excipient or vehicle.

The invention further provides a cell lysate of a cancer cell treated with a rfHSA of the invention.

A vaccine composition comprising a rfHSA-treated cancer cell's lysate is also provided. The vaccine composition may further comprise an adjuvant.

The cancer cell may be a cancer-derived cell line. In one embodiment, the cancer-derived cell line is from the same cancer as the subject's cancer or is the same type of cancer as the subject.

The invention also provides use of a rfHSA, a pharmaceutical composition, or a vaccine composition, of the invention in the manufacture of a medicament for treating cancer or for treating a tumor in a subject in need thereof.

In one embodiment, the cancer cell is at least one selected from the group consisting of brain cancer, breast cancer, cervical cancer, colorectal cancer, esophagus cancer, kidney cancer, liver cancer, larynx cancer, lung cancer, melanoma cancer, oral cancer, ovarian cancer, prostate cancer, pancreatic cancer, skin cancer, stomach cancer, testis cancer, and thyroid cancer cells.

The invention further provides use of a rfHSA of the invention in the manufacture of a reagent for detecting the presence of a cancer cell that is associated with integrin β1 or serine/threonine protein kinase Akt and extracellular signal-regulated kinase 1/2 (ERK 1/2) in tumor cells or in a tumor sample, or for inhibiting phosphorylation of Akt and ERK1/2 in a sample comprising a cancer cell.

The invention further provides a kit comprising a rfHSA of the invention for detecting the presence of a cancer cell that is associated with integrin β1 or Akt and ERK 1/2 in a tumor sample.

In another embodiment, prior to the use of a rfHSA of the invention in the manufacture of a medicament for treating cancer or for treating a tumor in a subject in need thereof, the use may further comprise use of a kit comprising the rfHSA of the invention for detecting the presence of a cancer cell that is associated with Akt and ERK1/2 in a tumor sample from the subject in need thereof.

The method for making the rfHSA of the invention comprises steps (a), (b), (c), (d), (e), (f), (g), (h), (i), and (j) as defined above.

In one embodiment, the concentration of the detergent in the eluent in eluting step (d) is lower than that in the buffer solution in dissolving step (a).

In another embodiment, performing dialysis step (g) may further comprise step (g′): replacing the dialysate comprising no detergent at least twice or three times with a fresh dialysate comprising no detergent.

In another embodiment, the detergent may be SDS.

In another embodiment, the size exclusion chromatography column has a pore size for separating proteins of 70 kDa molecular weight and above.

In another embodiment, the dialysate comprising no detergent is phosphate buffered saline.

According to the method of the invention, The repeating step in the method of the invention removes the detergent exhaustively, and the final concentrated, dialysis membrane eluate does not contain detectable fibrillar form of human serum albumin.

A method for treating cancer or a tumor in a subject in need thereof is also provided. The method comprises administering a therapeutically effective amount of the rfHSA, the pharmacological composition, or the vaccine composition, of the invention to the subject in need thereof.

Prior to administering the vaccine composition to the subject in need thereof, the method for treating may further comprise the step of treating a cancer cell line derived from the same cancer or same tissue type as the subject's with rfHSA of the invention.

### Examples

### Materials and Methods

Preparation of rfHSA. Twenty milligrams of clinical grade human serum albumin was dissolved in 10 ml of PBS with 1% SDS (w/v). The solution was sonicated for 5 min and subsequently applied to a SUPERDEX™-200, which was previously equilibrated with eluting solution (25 mM Tris-HCl (pH 8.0), 1 mM EDTA, 0.1 M NaCl, and 0.05% SDS). The column was eluted at the rate of 1 m/min and fractions C3 to C7 that contained human serum albumin were pooled. The pooled fractions were then dialyzed against PBS with CELLU-SEP® T4/Nominal (MWCO: 12,000-14,000 Da) dialysis membrane. New PBS buffer was exchanged every two hrs at room temperature (RT) three times. After dialysis against dialysate, the eluate inside dialysis tubing was collected, concentrated and dialyzed against dialysate again. The same procedure was repeated several times to remove the SDS as exhaustively as possible to obtain rfHSA of the invention. It was found that unlike fibrillar human serum albumin, rfHSA did not form fibrillar form.

Liquid chromatography-tandem mass spectrometry (LC-MS/MS). Naïve HSA and rfHSA proteins were analyzed and validated by high-performance liquid chromatography-tandem mass spectrometry (LC-MS/MS) on LTQ Orbitrap XL (THERMO FISHER SCIENTIFIC™, MA). In brief, the proteins were either limited proteolyzed with trypsin, or treated first with reducing agent tris(2-carboxyethyl)phosphine (TCEP; 20 mM), followed by alkylation of the free sulfhydryl groups with excess iodoacetamide (IAA) and then limited proteolyzed with trypsin. The resulting peptides were separated by high-performance liquid chromatography and the eluted peptides were ionized by nanospray ionization and analyzed in an on-line coupled LTQ Orbitrap XL mass spectrometer, using a top five collision-induced dissociation (CID) method with survey scans at 60,000 resolution and fragment ion detection in the ion trap operated at normal scan speed.

Cell survival determined by MTT colorimetric assay. Exponentially growing cells were seeded in 96-well plates in medium with 10%/6 FBS and incubated for 24 h. Treatment of cells with a series of concentrations of proteins was carried out in serum-free medium for 16 hrs. at 37° C. After treatment, MTT solution was added to each well (0.5 mg/ml), followed by a 4 hr incubation period. The viable cell number is directly proportional to the production of formazan, which, following solubilization with isopropanol, can be measured spectrophotometrically at 570 nm by an ELISA plate reader.

Effects of rfHSA on phosphorylation of Akt and ERK examined by western blotting. Cells were treated with or without different concentrations of rfHSA. Cellular extracts (40-60 μg) of cancer samples were loaded onto two SDS-polyacrylamide gels as indicated. After gel electrophoresis, the proteins on the gels were transferred to two PVDF membranes. The proteins on two membranes were cut according to their molecular weights to make chopped blots. Each chopped blot was labeled to indicate which antibodies would be used for staining. The chopped blots were blocked with 5% non-fat milk at RT for 1 hr, thoroughly washed with 1×Tris-buffered saline and TWEEN® 20 (TBST), and treated with respective primary antibodies at 4° C. overnight. The chopped blots were thoroughly washed with 1×TBST, treated with peroxidase labeled secondary antibodies at RT for 1 hr and then thoroughly washed with 1×TBST. The blots were treated with peroxidase substrate for enhanced chemiluminescence (ECL). The blots were then detected on BIOMAX® ML films with a KODAK® medical x-ray processor, scanned and put together as a finished graph in the computer.

It

B16F10 vaccination experiments. For rfHSA-induced immunogenic cell death (ICD) total cell lysates (TCLs), B16F10 melanoma cells were treated with 1.5 μM rfHSA for 24 hrs. to induce ICD. After scraping, centrifuging and washing with PBS twice, 1×10⁷ B16F10 per milliliter were suspended in PBS and then repeatedly freeze-thawed four times. After centrifugation, the supernatants were collected and stored for vaccination. For vaccination, C57BL/6 mice were subcutaneously injected with 100 μl of rfHSA-induced ICD TCLs from B16F10 cells into the left flank only once during 2 weeks as the prime group and once a week for 2 weeks as the boost group, respectively. One week after the final vaccination, the right flank was subcutaneously injected with 1×10⁴ of the same live B16F10 cells. Tumor formation was monitored. Tumor volume was immediately recorded on the indicated day and was calculated according to the following formula: volume=(length×width²×π)/6 until sacrifice at 32 days after injecting live cancer cells. The survival rate and number of tumor-free mice were recorded. Tumor weights were immediately recorded on the indicated day after sacrifice.

### Results

rfHSA Exhibiting an Oval Shape and Naive HSA a Globular Shape in Solution as Indicated by Biological Small Angel x-Ray Scattering.

Small angel x-ray scattering was used to analyze the structures of rfHSA and naïve HSA. FIGS. 1A-B illustrate the model docking of 1e78.pdb crystal structures, rfHSA envelope and naïve HSA envelop using the program SUPCOMB (M. Kozin & D. Svergun “Automated matching of high- and low-resolution structural models” J Appl Cryst. 2001, 34: 33-41). The results indicate that the shape of rfHSA is different from that of naïve HSA. The shape of rfHSA is oval rather than globular as naïve HSA.

Liquid Chromatography-Tandem Mass Spectrometry Analysis after Limited Proteolysis Under Non-Reducing Condition Indicates that rfHSA is Different from Naïve HSA.

The protein made as described above is mainly rfHSA, instead of a mixture of rfHSA and naïve HSA.

Under a reducing condition using tris(2-carboxyethyl)phosphine (TCEP; 20 mM) to reduce disulfide bridges, followed by alkylation of free sulfhydryl group with iodoacetamide, it was found that rfHSA mass spectrum after limited proteolysis with trypsin (50 μg/ml for 1 min) was similar to that of naïve HSA (FIGS. 2A-B).

Under a non-reducing condition, rfHSA mass spectrum after limited proteolysis with trypsin was different from that of naïve HSA, notably in the lack of three major fragment ions at m/z 4559, 5729 and 7223 (FIGS. 3A-B). The lack of these three major fragment ions in rfHSA indicates that rfHSA is distinguishable from naïve HSA and there is very little or no naïve HSA in our preparation batch of rfHSA. The native conformation of naïve HSA is primarily preserved by 17 intramolecular disulfide bridges. Under reducing condition, rfHSA mass spectra after limited proteolysis with trypsin, like those of naïve HSA, shows parent ion at m/z 3030 and several fragment ions notably those at m/z 2706, 2259, 2044 and 1148. The three major fragment ions at m/z 4559, 5729 and 7223 appearing under non-reducing condition for naïve HSA have mass to charge ratio greater than 3030 are most likely peptide fragment ions with disulfide bonds.

rfHSA does not have Disulfide Bridges that Link Cysteine 124 to Cysteine 168 and Cysteine 169 to Cysteine 177.

The tryptic digests of naïve HSA were separated and fractionated by preparative liquid chromatography. Mass spectrometry sequencing was used to confirm the peptide sequence corresponding to fragment ion at m/z 4559. The result indicates that the fragment ion at m/z 4559 is a 41 amino acid peptide with the amino acid sequence of LVRPEVDVMC($1)TAFHDNEETFLK AAFT EC($1)C($2)QAADKAA C($2)LLPK (SEQ ID NO: 2). Search of the amino acid sequence of naïve HSA revealed that this peptide is formed by the disulfide bridges that link cysteine 124 to cysteine 168 and cysteine 169 to cysteine 177 (FIGS. 4A-B). Since the fragment ion at m/z 4559 is present in naïve HSA and absent in rfHSA, the result demonstrates that rfHSA does not have disulfide bridges that link cysteine 124 to cysteine 168 and cysteine 169 to cysteine 177.

rfHSA does not have Disulfide Bridges that Link Cysteine 567 to Cysteine 75 and Cysteine 62 to Cysteine 361.

The tryptic digests of naïve HSA were separated and fractionated by preparative liquid chromatography. Tandem mass spectrometry sequencing was used to confirm the peptide sequence corresponding to fragment ion at m/z 5729. Our result shows that the fragment ion at m/z 5729 is a 53 amino acid polypeptide with sequence of KGEEAFC($1)TEK LTAVTC($1)LKDGFLTHLSKDC($2)NEASEDAVCTKAYCEHPDAAAC($2)CK (SEQ ID NO: 3). Search of the amino acid sequence of naïve HSA revealed that this polypeptide is formed by the disulfide bridges that link cysteine 567 to cysteine 75 and cysteine 62 to cysteine 361 of HSA sequence (FIGS. 5A-B). Since the fragment ion at m/z 5729 is present in naïve HSA and obscured in rfHSA, the result demonstrates that majority of rfHSA does not have disulfide bridges that link cysteine 567 to cysteine 75 and cysteine 62 to cysteine 361.

rfHSA does not have Disulfide Bridges that Link Cysteine 487 to Cysteine360 and Cysteine 361 to Cysteine 62.

The tryptic digests of naïve HSA were separated and fractionated by preparative liquid chromatography. Tandem mass spectrometry sequencing was used to confirm the peptide sequence corresponding to fragment ion at m/z 7223. Our result shows that the fragment ion at m/z 7223 is a 65 amino acid polypeptide with sequence of VTKCCTESLVNRRPC($1)FSALEVDETYVPK LAKTYETTLEKC (S1)C(S2)AAADPHECYAK TCVADESAENC($2)DK (SEQ ID NO: 4). Search of the amino acid sequence of naïve HSA revealed that this peptide is formed by the disulfide bridges that link cysteine 487 to cysteine360 and cysteine 361 to cysteine 62 (FIGS. 6A-B). Since the fragment ion at m/z 7223 is present in naïve HSA and absent in rfHSA, the result demonstrates that rfHSA does not have disulfide bridges that link cysteine 487 to cysteine360 and cysteine 361 to cysteine 62.

rfHSA is Cytotoxic to a Variety of Cancer Cells

The respective cell types were treated with various concentrations of rfHSA for 16 hrs. in serum-free culture medium. Cell viability was examined by MTT cell proliferation assay and IC₅₀ of rfHSA on the viability of each cancer cell line was determined. FIG. 7 illustrates the half maximal inhibitory concentrations (IC₅₀) of rfHSA on a variety of cancer cells.

rfHSA has Cytotoxic Effects on Clinically Relevant Ovarian Cancer Cell Lines

The cell cytotoxicity of rfHSA on ovarian adenocarcinoma cell lines TOV-21G, OVSAHO and ovarian carcinoma KURAMOCHI cell lines were investigated (FIG. 8, top panel). The cells were treated with rfHSA and cell viabilities examined by MTT cell proliferation assay. The viabilities of the cells were plotted against the concentrations of rfHSA (FIG. 8, bottom panel).

rfHSA Suppresses Ovarian Cancer Growth In Vivo.

FIG. 9 show that rfHSA was effective in suppressing tumor cell proliferation in an intraperitoneal (I.P.) ovarian murine model. Ovarian cancer SKOV3 cells pre-labelled with green fluorescent protein and firefly luciferase (SKOV3-GL) were administered into nude mice I.P. and control vehicle or rfHSA (15 mg/kg) was then injected once every week as indicated (FIG. 9A). Bioluminescence imaging (BLI) and measurement of body weight revealed that rfHSA significantly reduced proliferation of the tumor cells without affecting mouse body weight (FIGS. 9B-C).

rfHSA has Cytotoxic Effect on Pancreatic Cancer Cell Lines.

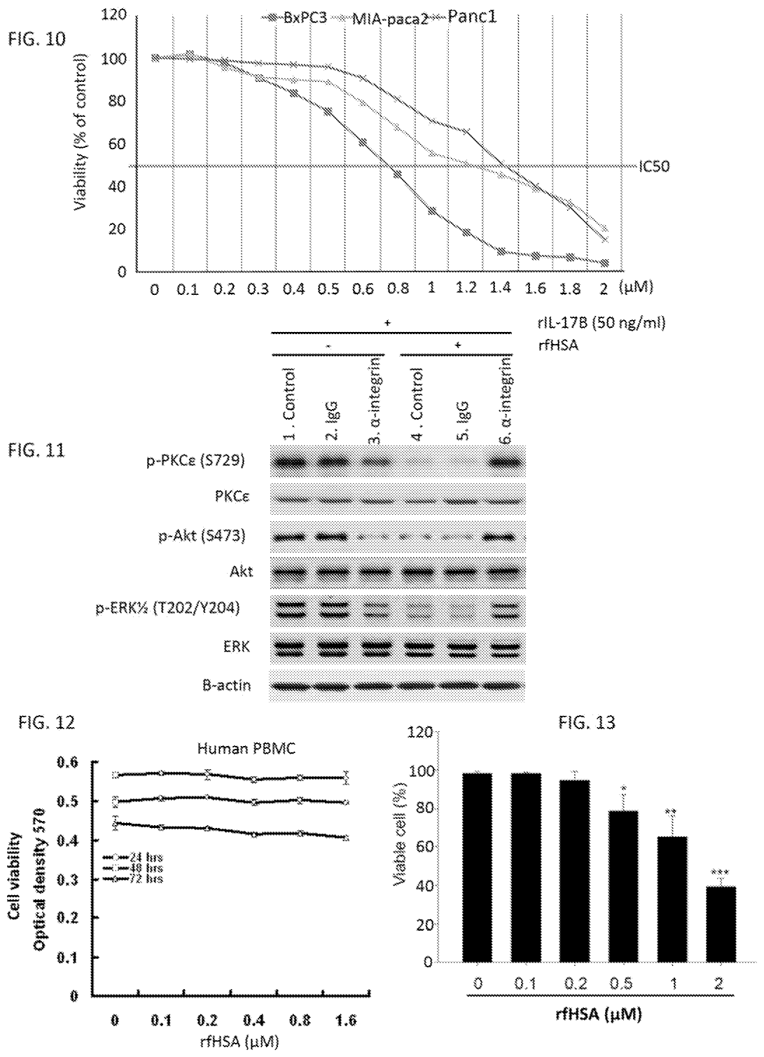

FIG. 10 shows that rfHSA suppressed pancreatic cancer cell lines BxPC3, MIA-paca2 and Panc1, respectively, in vitro. The cells were treated with rfHSA and cell viabilities were examined by MIT cell proliferation assay as described previously. The viabilities of the cells were plotted against the concentrations of rfHSA.

rfHSA Inhibits Phosphorylation of Akt and ERK1/2 of Pancreatic Cancer Cells in an Integrin β1-Dependent Manner.

FIG. 11 shows that anti-integrin β1 antibodies reversed the inhibitory effects of rfHSA on phosphorylation of Akt and ERK1/2. BxPC3 cells were pretreated with control IgG or anti-integrin β1 antibodies (2 μg/ml) for 30 min followed by IL17B (50 ng/ml) or/and rfHSA (0.2 μM) treatment for 24 hrs. in 0.1% FBS medium. The expression levels of phospho-Akt, total Akt, phospho-ERK1/2 and total ERK1/2 were determined by western blot. β-actin was used as a loading control. Blots are representative of three independent experiments.

rfHSA is not Cytotoxic to Normal Cells.

FIG. 12 shows that rfHSA did not induce cell death in human primary peripheral blood mononuclear cells (PBMC). Human PBMC were treated with serial concentrations of rfHSA for 24, 48 and 72 hrs as indicated. Cell viability was examined by the MTT cell proliferation assay and the viability of the cell was plotted versus the concentration of rfHSA.

rfHSA Induces Cytotoxic Effect on B16F10 Melanoma Cancer Cells.

B16F10 melanoma cells were treated with different amounts of rfHSA for 24 hrs. The cells were harvested to measure the cell survival rate by counting the percentage of viable cells using trypan blue staining. FIG. 13 shows rfHSA inhibited cell viability in a dose-dependent manner.

Vaccination with rfHSA-Treated B16F10 Cell Lysate Elicits Anti-Tumor Immune Response and Tumor Clearance In Vivo.

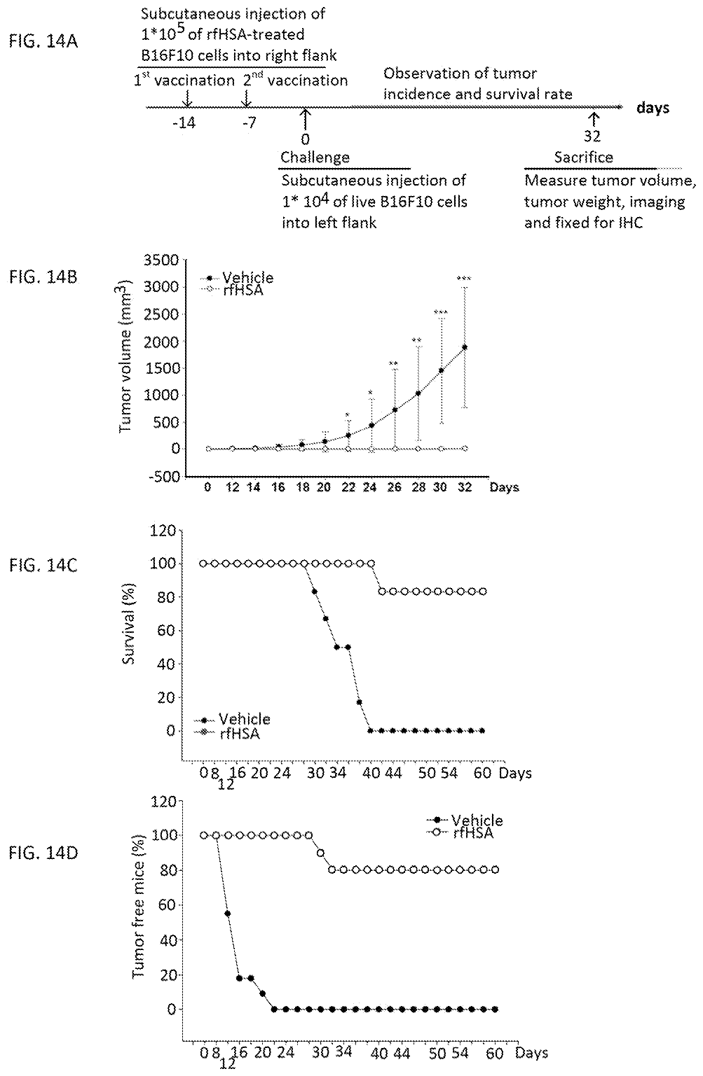

Cancer cells were treated with rfHSA for 24 hrs. and the cytosolic lysate was inoculated into mice and boosted again one week later. The mice were then injected with live B16F10 melanoma cells (1×10⁴ cells/mouse). FIGS. 14A-D shows vaccination with rfHSA-treated B16F10 cell lysate elicited anti-tumor immune response and tumor clearance in vivo. (A) The schematic vaccination protocol of this study. The treatment of rfHSA (1.5 μM) for 24 hers was performed. After challenge, (B) the tumor growth rate, (C) mouse survival rate and (D) tumor-free outcomes were monitored. The tumor volume and survival rate of the mice with cytosolic lysate vaccination were found to much less tumor and survived significantly longer.

1. A re-folded human serum albumin (rfHSA) molecule, the rfHSA molecule comprising the primary amino acid sequence of naïve human serum albumin (naïve HSA) molecule, wherein the rfHSA molecule in a solution is oval shape, not fibrillar, and the naïve HSA molecule is globular.

2. The rfHSA molecule of claim 1, which lacks two or more intramolecular disulfide bridges selected from the group consisting of:
(i) C124-C168 and C169-C177;
(ii) C567-C75 and C62-C361;
(iii) C487-C360 and C361-C62; and
(iv) any combination thereof.

3. The rfHSA molecule of claim 1, which lacks the intramolecular disulfide bridges C124-C168, C169-C177, C567-C75, C62-C361, and C487-C360.

4. The rfHSA molecule of claim 1, which after limited trypsin proteolysis under nonreduced conditions lacks mass spectrum fragment ions at mass to charge ratios (m/z) of 4559, 5729 and 7223, wherein the fragment ions at the m/z of 4559, 5729 and 7223 are present after the limited trypsin proteolysis of the naïve HSA.

5. The rfHSA molecule of claim 1, which after limited trypsin proteolysis under nonreduced conditions generates peptide fragments, the generated fragments lacking one or more peptide fragments comprising an amino acid sequence selected from the group consisting of SEQ ID NOs: 2, 3, 4, and any combination thereof, wherein the lacked one or more peptide fragments are present after the limited trypsin proteolysis of the naïve HSA molecule.

6. The rfHSA molecule of claim 1, which after limited trypsin proteolysis under nonreduced conditions generates peptide fragments, the generated fragments lacking peptide fragments comprising the amino acid sequence of SEQ ID NOs: 2, 3, and 4, respectively, wherein the lacked peptide fragments are present after the limited trypsin proteolysis of the naïve HSA molecule.

7. A method for treating cancer, inhibiting cancer cell growth, inducing cancer cell cytotoxicity, and/or increasing survival rate in a subject in need thereof, comprising the step of:
administering a therapeutically effective amount of the rfHSA molecule of claim 1 to the subject in need thereof.

8. The method of claim 7, wherein the cancer is at least one selected from the group consisting of brain cancer, breast cancer, cervical cancer, colorectal cancer, esophagus cancer, kidney cancer, liver cancer, larynx cancer, lung cancer, melanoma cancer, oral cancer, ovarian cancer, prostate cancer, pancreatic cancer, skin cancer, stomach cancer, testis cancer, and thyroid cancer.

9. (canceled)

10. (canceled)

11. A kit comprising the rfHSA molecule of claim 1 for detecting the presence of a cancer cell that is associated with integrin β1 or serine/threonine protein kinase Akt and extracellular signal-regulated kinase 1/2 (ERK1/2) in tumor cells or a tumor sample, or for inhibiting phosphorylation of Akt and ERK1/2 in a sample comprising a cancer cell.

12. The method of claim 7, prior to the administering step further comprising the step of:
providing a kit comprising the rfHSA molecule for detecting the presence of a cancer cell that is associated with Akt and ERK1/2 in a tumor sample from the subject in need thereof.

13. A cell lysate of a cancer cell treated with the rfHSA molecule of claim 1.

14. The cell lysate of claim 13, wherein the cancer cell is at least one selected from the group consisting of brain cancer, breast cancer, cervical cancer, colorectal cancer, esophagus cancer, kidney cancer, liver cancer, larynx cancer, lung cancer, melanoma cancer, oral cancer, ovarian cancer, prostate cancer, pancreatic cancer, skin cancer, stomach cancer, testis cancer, and thyroid cancer cells.

15. A vaccine composition comprising the cell lysate of claim 13.

16. The vaccine composition of claim 15, further comprising an adjuvant.

17. A method for treating a tumor or for inhibiting growth of a tumor in a subject in need thereof, comprising the step of:
administering a therapeutically effective amount of the vaccine composition of claim 15 to the subject in need thereof.

18. A method for making the rfHSA molecule of claim 1, comprising:
(a) dissolving human serum albumin (HSA) in a buffer solution comprising a detergent to obtain a detergent-treated HSA solution;
(b) sonicating the detergent-treated HSA solution to obtain a sonicated, detergent-treated HSA solution;
(c) subjecting the sonicated, detergent-treated HSA solution to a size exclusion chromatography column with a molecular weight range between 10,000 and 600,000 Daltons (Da);
(d) eluting the column with an eluent comprising the detergent;
(e) collecting column eluate fractions comprising the detergent-treated HSA;
(f) pooling the column eluate fractions to obtain a pooled column eluate;
(g) performing dialysis by subjecting the pooled column eluate to a dialysis membrane with molecular weight-cutoff (MWCO) of 12,000-14,000 Da against a dialysate comprising no detergent;
(h) collecting a dialysis membrane eluate;
(i) concentrating and dialyzing the dialysis membrane eluate against the dialysate comprising not detergent to obtain a concentrated, dialysis membrane eluate; and
(j) repeating the concentrating and dialyzing step (i) to obtain a final concentrated, dialysis membrane eluate comprising the rfHSA of claim 1, wherein the final concentrated, dialysis membrane eluate in step (j) comprises no or little detergent.

19. The method of claim 18, wherein the concentration of the detergent in the eluent in eluting step (d) is lower than that in the buffer solution in dissolving step (a).

20. The method of claim 18, wherein the performing dialysis step (g) further comprises:
(g′) replacing the dialysate comprising no detergent at least twice or three times with a fresh dialysate comprising no detergent.

21. A method for treating cancer, inhibiting cancer cell growth, inducing cancer cell cytotoxicity, and/or increasing survival rate in a subject in need thereof, comprising the step of:
administering a therapeutically effective amount of the rfHSA molecule of claim 2 to the subject in need thereof.

22. A method for treating a tumor or for inhibiting growth of a tumor in a subject in need thereof, comprising the step of:
administering a therapeutically effective amount of the vaccine composition of claim 16 to the subject in need thereof.
